(19) United States
(12) Patent Application Publication
(10) Pub. No.: US 2006/0148868 A1
(43) Pub. Date: Jul. 6, 2006

(54) Zolmitriptan polymorphs

(76) Inventors: Venkataraman Sundaram, Hyderabad (IN); Purandhar Koilkonda, Hyderabad (IN); Amarnath Reddy Lekkala, Hyderabad (IN); Vijaykumar Kotagiri, Hyderabad (IN); Sashikanth Suthrapu, Warangal (IN); Kondaiah China Mala Golla, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/284,729

(22) Filed: Nov. 22, 2005

Related U.S. Application Data
(60) Provisional application No. 60/630,285, filed on Nov. 23, 2004.
(60) Provisional application No. 60/673,141, filed on Apr. 20, 2005.

(30) Foreign Application Priority Data
Mar. 8, 2005 (IN) 226/CHE/2005

(51) Int. Cl. A61K 31/422; C07D 413/02

(52) U.S. Cl. 514/376; 548/229

(57) ABSTRACT

Crystalline polymorphic forms of zolmitriptan, solid amorphous zolmitriptan, and processes for preparing them.

### CROSS-REFERENCE TO RELATED APPLICATIONS

This application is a nonprovisional filing of U.S. Provisional Applications 60/630,285 filed Nov. 23, 2004, and 60/673,141 filed Apr. 20, 2005, the entire disclosures of which are hereby incorporated by reference.

### INTRODUCTION TO THE INVENTION

The present invention relates to polymorphic crystalline Form II and Form III of zolmitriptan, a solid amorphous form of zolmitriptan, and processes for their preparation.

Zolmitriptan has the chemical name (S)-4-[[3-[2-(dimethylamino)ethyl]-1H-indol-5-yl]-methyl]-2-oxazolidinone and is structurally represented by Formula I.

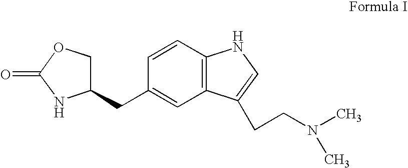

Zolmitriptan is a selective 5-hydroxytryptamine1B/1D (5-HT_{1B/1D}) receptor agonist and is of particular use in the treatment of migraine and associated conditions. Commercial products comprising zolmitriptan are available under the name ZOMIG™ in the forms of tablets, orally disintegrating tablets, and nasal spray.

U.S. Pat. No. 5,466,699 describes processes for the preparation of zolmitriptan in Examples 2 and 3. These processes result in the preparation of zolmitriptan isopropanolate hemihydrate.

U.S. Pat. Nos. 6,084,103 and 6,160,123 describe a one-pot process for the preparation of zolmitriptan that yields zolmitriptan ethyl acetate solvate, subsequently treated in an acetone water mixture to remove ethyl acetate.

These patents do not describe the crystallinity of the products or the characterization of the solid state properties.

PCT International Publication No. 2005/075467 describes various crystalline forms of zolmitriptan and gives processes for their preparation. The application describes the formation of several crystalline forms of zolmitriptan, which were designated zolmitriptan Form A, the 1-butanol solvate of zolmitriptan designated as Form B, the anisole solvate of zolmitriptan designated as Form C, the 2-propanol solvate of zolmitriptan designated as Form D, the ethyl methyl ketone solvate of zolmitriptan designated as Form E, the tetrahydrofuran solvate of zolmitriptan designated as Form F, and the 1,4-dioxane solvate of zolmitriptan designated as Form G. Also disclosed is an amorphous zolmitriptan, obtained as an oil from evaporation of a solution of zolmitriptan, which can be used to prepare Form A.

It is known that different polymorphic forms of the same drug may have substantial differences in certain pharmaceutically important properties. The amorphous form of drug may exhibit different dissolution characteristics and in some cases different bioavailability patterns compared to crystalline forms. For some therapeutic indications one bioavailability pattern may be favored over another. Further, amorphous and crystalline forms of a drug may have different handling properties, dissolution rates, solubility, and stability. For these reasons, among others, access to a choice between the amorphous or different crystalline forms of a drug is desirable for various applications. Therefore, regulatory authorities desire to have all possible polymorphic forms of a new drug substance identified prior to approval of a product containing the drug.

However, as is well known in the art, the existence of polymorphic forms of any given compound cannot be predicted, and there is no standard procedure for proceeding to make a previously unknown polymorphic form. Even after a polymorph has been identified, there is no possibility of predicting whether any additional forms will ever be discovered. This situation has been the subject of recent articles, including A. Goho, “Tricky Business,” Science News, Vol. 166, No. 8, pages 122-123 (August 2004).

Therefore, there is a continuing need to prepare new polymorphic forms of pharmacologically active compounds of commercial interest which provide the pharmaceutical formulation scientist with a broader spectrum of forms of an active ingredient to choose from, based on their differing properties.

### SUMMARY OF THE INVENTION

The present invention relates to crystalline Form II and Form III of zolmitriptan and solid amorphous zolmitriptan, and processes for their preparation.

In one aspect, crystalline Form II of zolmitriptan is prepared by a method comprising evaporation of solvent from a solution of zolmitriptan in a solvent comprising a halogenated hydrocarbon.

In another aspect, crystalline Form III of zolmitriptan is prepared by a method comprising suspending zolmitriptan in an aromatic hydrocarbon, adding a lower alcohol to form a solution, and crystallizing Form III of zolmitriptan.

In a further aspect, solid amorphous zolmitriptan is prepared by a method comprising cooling a solution of zolmitriptan in a halogenated hydrocarbon solvent to a temperature where precipitation of amorphous zolmitriptan occurs.

### BRIEF DESCRIPTION OF THE DRAWINGS

FIG. 1 is an X-ray powder diffraction pattern of crystalline Form II of zolmitriptan.

FIG. 2 is an X-ray powder diffraction pattern of crystalline Form III of zolmitriptan.

FIG. 3 is an X-ray powder diffraction pattern of solid amorphous zolmitriptan.

### DETAILED DESCRIPTION

The present invention relates to crystalline Form II and Form III and the solid amorphous form of zolmitriptan, and to processes for their preparation.

The various forms of Zolmitriptan are characterized by characteristic X-ray powder diffraction (“XRPD”) patterns. All XRPD results reported or claimed herein are obtained using Cu Ka radiation, having the wavelength 1.541 Å. The XRPD patterns of the figures have a vertical axis that is intensity units, and a horizontal axis that is the 2θ angle, in degrees.

The locations of the XRPD peaks (i.e., the 2θ angles) are generally quite constant for a given crystalline substance, subject to small variations due to instrument tolerances. However, the heights of individual peaks can vary considerably between samples of the same substance, frequently because of sample preparation differences. Therefore, when comparing XRPD patterns, the peak locations, and not their relative intensities, are better indicators of whether the same substances are present in the samples.

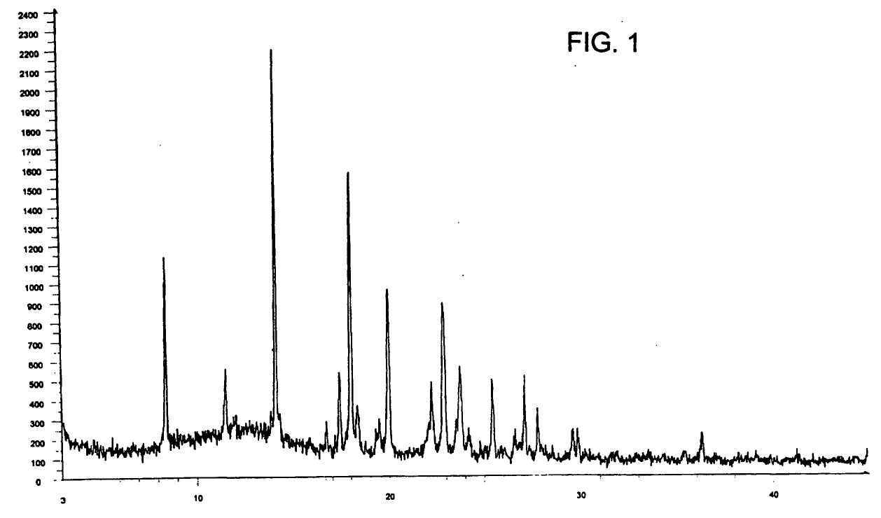

Crystalline zolmitriptan Form II exhibits an X-ray powder diffraction pattern substantially according to the pattern of FIG. 1 with characteristic peaks expressed in d-values (Å) and in 2θ angles as given in the following Table 1.

TABLE 1

| 2θ Angle (degrees) | d-spacing (Å) | Qualitative Relative Intensity |
|---|---|---|
| 8.3 | 10.58 | Strong |
| 11.3 | 7.77 | Weak |
| 11.8 | 7.46 | Very weak |
| 13.7 | 6.43 | Very weak |
| 14.0 | 6.31 | Very strong |
| 14.2 | 6.20 | Very weak |
| 16.6 | 5.32 | Very Weak |
| 17.3 | 5.10 | Medium |
| 17.9 | 4.93 | Very strong |
| 18.2 | 4.84 | Weak |
| 19.2 | 4.60 | Very weak |
| 19.4 | 4.56 | Very weak |
| 19.9 | 4.45 | Strong |
| 21.8 | 4.07 | Very weak |
| 22.1 | 4.00 | Medium |
| 22.8 | 3.89 | Strong |
| 23.4 | 3.78 | Very weak |
| 23.7 | 3.75 | Medium |
| 24.1 | 3.68 | Very weak |
| 25.3 | 3.50 | Medium |
| 26.5 | 3.35 | Very weak |
| 27.0 | 3.29 | Medium |
| 27.7 | 3.21 | Weak |
| 29.5 | 3.02 | Very weak |
| 29.8 | 2.99 | Very weak |
| 31.8 | 2.80 | Very weak |
| 35.3 | 2.53 | Very weak |
| 36.2 | 2.47 | Very weak |

Crystalline Form II of zolmitriptan can be distinguishably characterized by significant XRPD peaks at about 8.3, 11.8, 14.0, 17.3, 19.9, 22.1, 22.8, 23.7, and 25.3±0.2 degrees 2θ. Form II can be further characterized by, the additional XRPD peaks at about 27.0, 27.7, 29.5, and 36.2, ±0.2 degrees 2θ.

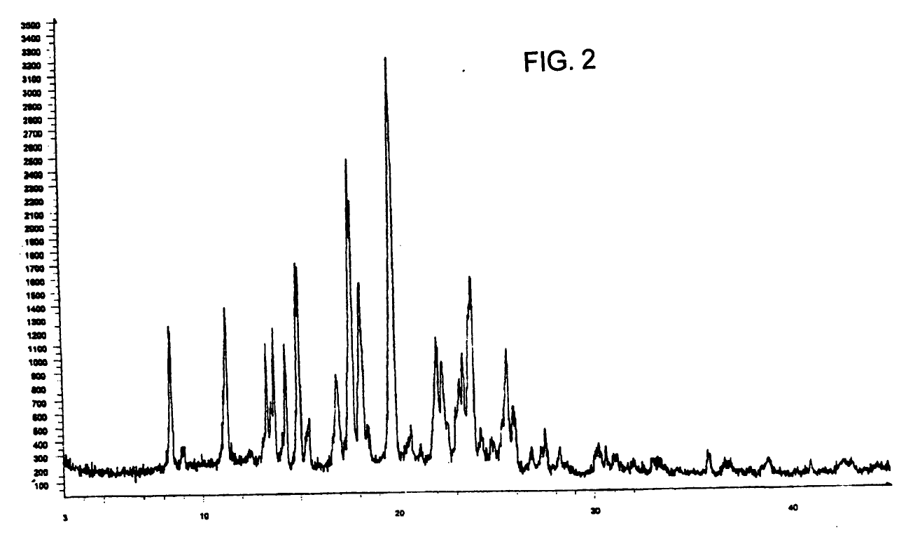

Crystalline zolmitriptan Form III exhibits a characteristic X-ray powder diffraction pattern substantially according to the pattern of FIG. 2, with characteristic peaks expressed in d-values (Å) and in 2θ angles as given in the following Table 2.

TABLE 2

| 2θ Angle (degrees) | d-spacing (Å) | Qualitative Relative Intensity |
|---|---|---|
| 8.3 | 10.6 | Strong |
| 8.9 | 9.89 | Very weak |
| 11.1 | 7.93 | Strong |
| 13.2 | 6.67 | Medium |
| 13.6 | 6.49 | Medium |
| 14.2 | 6.22 | Medium |
| 14.8 | 5.94 | Strong |
| 15.3 | 5.76 | Weak |
| 16.8 | 5.25 | Medium |
| 17.5 | 5.05 | Very strong |
| 18.0 | 4.91 | Strong |
| 18.4 | 4.81 | Very weak |
| 19.6 | 4.50 | Very strong |
| 20.6 | 4.30 | Very weak |
| 21.9 | 4.04 | Medium |
| 22.2 | 3.98 | Medium |
| 22.5 | 3.94 | Very weak |
| 23.3 | 3.81 | Medium |
| 23.7 | 3.74 | Strong |
| 24.2 | 3.67 | Very weak |
| 24.7 | 3.59 | Very weak |
| 25.5 | 3.48 | Medium |
| 25.8 | 3.44 | Weak |
| 26.7 | 3.32 | Very weak |
| 27.2 | 3.26 | Very weak |
| 27.4 | 3.24 | Very weak |
| 28.1 | 3.16 | Very weak |
| 30.1 | 2.95 | Very weak |
| 30.5 | 2.92 | Very weak |
| 32.9 | 2.71 | Very weak |
| 35.7 | 2.50 | Very weak |
| 38.8 | 2.31 | Very weak |

Crystalline Form III of zolmitriptan can be distinguishably characterized by significant XRPD peaks at about 8.3, 11.1, 14.8, 15.3, 17.5, 18.0, 18.4, 19.6, 23.7, and 25.5, ±0.2 degrees 2θ. Form III can be further characterized by the additional XRPD peaks at about 13.2, 13.6, 23.3, 25.8, and 27.4, ±0.2 degrees 2θ.

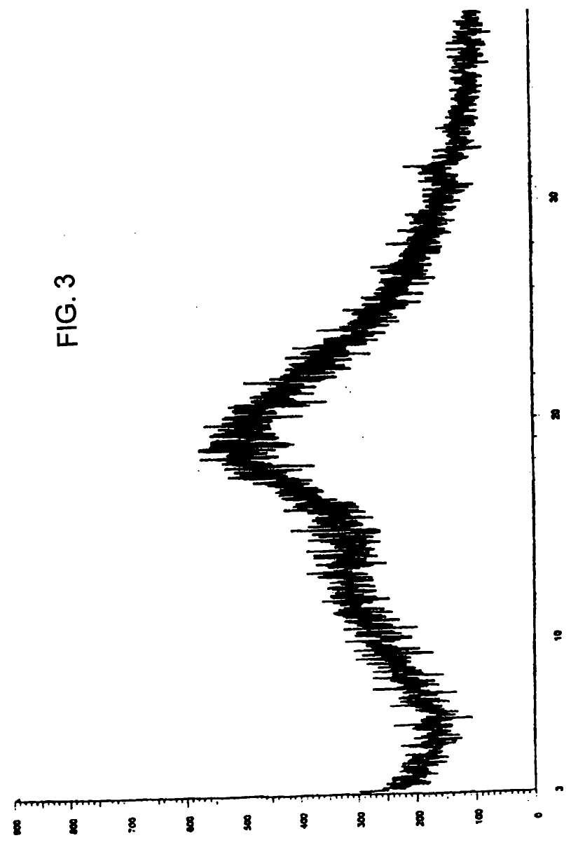

Solid amorphous zolmitriptan has an XRPD pattern substantially as shown in FIG. 3, showing a halo which is a characteristic of the amorphous nature of the substance.

In an aspect, the present invention provides a process for the preparation of crystalline Form II of zolmitriptan comprising evaporation of the solvent from a solution of zolmitriptan in a solvent comprising a halogenated hydrocarbon.

Suitable halogenated hydrocarbon solvents include, but are not limited to dichloromethane, chloroform, carbon tetrachloride, and the like, and mixtures of any two or more thereof. The quantity of solvent used for dissolution can range from 10 to 100 times to the zolmitriptan taken or 10 to 20 times to the quantity of zolmitriptan taken. Dissolution can be achieved at ambient temperatures or at higher temperatures ranging from about 35 to 75° C. or about 35 to 55° C. Generally, higher temperatures will permit the use of less solvent, reducing the volume that must be evaporated.

The solution can optionally be filtered to remove any undissolved material, either in the hot condition or after cooling to lower temperatures, which temperatures should be sufficiently high to maintain the solution. The filtration may be achieved by passing the solution through paper, glass fiber or other membrane material, or a bed of a clarifying agent such as celite. The filter media may further be washed with the solvent used for dissolution, or some other solvent.

The evaporation of the solvent can be performed by distillation at atmospheric or at reduced pressures, lower temperatures generally being required as the pressure is reduced. The distillation can be performed at a temperature typically above 35° C. in a Buchi Rotovapor, or other techniques for evaporation of solvent can be adopted such as for example, spray drying, agitated thin film drying and the like.

The obtained compound can optionally be further dried under ambient or reduced pressure. For example, drying can be performed under reduced pressure or under atmospheric pressure at temperatures typically about 40° C. to 60° C., or 70° C. to 80° C., or higher temperatures that do not affect stability of the compound.

In another aspect, the present invention provides a process for the preparation of crystalline Form III of zolmitriptan comprising the steps of:

a) suspending zolmitriptan in a suitable solvent;

b) adding another solvent to form a solution; and

c) cooling the solution to crystallize Form III of zolmitriptan.

Solvents suitable for step a) include aromatic hydrocarbons such as toluene and xylene, and the like, or mixtures thereof.

Solvents suitable for step b) include solvents such as, for example, lower alcohols having 1 to about 4 carbon atoms, like methanol, ethanol, isopropanol, and n-butanol.

The quantity of solvent used for dissolution can range from 10 to 100 times to the zolmitriptan taken or 10 to 20 times to the quantity of zolmitriptan taken. The ratio of the mixture of solvents used may range anywhere from 1:20 to 20:1. Dissolution will be achieved at temperatures higher than the temperatures that will be used for crystallization, such as ranging from about 35 to 75° C., or about 35 to 55° C.

The crystallization is performed with stirring and can be performed at about 20° C. to about 25° C., or at higher or lower temperatures, until the desired crystal yield has been achieved, such as for about 1 to about 72 hours. The crystallization step may further include cooling the solution as required.

Recovery of the crystals can be achieved by any means including but not limited to filtration, centrifugation and decanting.

The crystalline form may be recovered from any composition containing the crystalline form and the solvent or solvents including, but not limited to, a suspension, solution, slurry, and emulsion.

The obtained crystals can optionally be further dried under ambient or reduced pressure. For example, drying can be performed under reduced pressure or under atmospheric pressure at a temperature of about 40° C. to 60° C., or 70° C. to 80° C., or higher temperatures that do not affect stability of the compound. Drying can be performed for any desired time, but usually a drying time of about 2 to 24 hours, or about 3 to 6 hours, is sufficient.

In a further aspect, the present invention provides a process for the preparation of solid amorphous zolmitriptan comprising the steps of:

a) dissolving zolmitriptan in a suitable halogenated hydrocarbon solvent at an elevated temperature; and

b) cooling the solution to a temperature where precipitation of the compound occurs.

Suitable halogenated hydrocarbon solvents include but are not limited to dichloromethane, chloroform, carbon tetrachloride and the like and mixtures of any two or more thereof.

The quantity of solvent used for dissolution can range from about 50 to 150 times, or about 50 to 70 times, the quantity of zolmitriptan taken.

The solution obtained in step a can optionally be filtered either in the hot condition or after cooling to lower temperatures that maintain the solution, by passing through paper, cloth, or other membrane material, or a bed of a clarifying agent such as celite. Filtration can be carried out more than once to ensure that the filtrate is clear. The filter media can further be washed with the solvent used for dissolution or some other solvent may be used.

Precipitation can be performed with stirring at ambient or reduced temperatures such as at about 20° C. to about 25° C., for a time that provides the desired product yields, such as about 1 to about 72 hours. The crystallization step can further include cooling the solution as required.

Recovery of the precipitated solid can be performed by any means including but not limited to filtration, centrifugation and decanting.

The obtained solid can be further dried under ambient or reduced pressure. For example, drying can be performed under reduced pressure or under atmospheric pressure at a temperature of at about 40° C. to 60° C., or 70° C. to 80° C., or higher temperatures that do not affect stability of the compound. Drying can be performed for any desired time, but usually a sufficient drying time is about 2 to 24 hours, or about 3 to 6 hours.

In all the above mentioned processes, the solution can be prepared by dissolving zolmitriptan in a solvent. Any polymorphic form of zolmitriptan can be used for the preparation of the solution in this invention. This can include any crystalline or amorphous form of zolmitriptan including any salts, solvates, and hydrates. The amount of solvent to be used to prepare the solution should be sufficient to dissolve the zolmitriptan. Preferably, the dissolving step further includes stirring the solution, which can be achieved by any means including but not limited to mechanical and magnetic means. The dissolving step can further include facilitative measures known to one skilled in art. For example, the dissolving step may further include heating, filtering, and/or diluting the solution.

If warming does not form a completely clear solution, the mixture can be diluted or solids can be separated using techniques such as filtration, centrifugation, decantation, etc. To filter, a hot mixture can be passed through paper, glass fiber or other membrane material, or a bed of a clarifying agent such as celite. Depending upon the equipment used and the concentration and temperature of the solution, the filtration apparatus may need to be preheated to avoid premature crystallization or precipitation.

Preferably, the solid formation step is performed with stirring. The solid formation step can be performed at about 20° C. to about 25° C. or at elevated temperature, such as about 25 to 60° C., or at a lower temperature, such as about 0° C. to about 20° C. The solid formation step can further include facilitative measures known to one skilled in the art. For example, a crystallization step may further include cooling the solution, heating the solution, or adding an agent to induce precipitation.

Recovering the crystalline or amorphous zolmitriptan can be performed by any means known in the art including, but not limited to filtration, centrifugation, and decanting. The crystalline form may be recovered from any composition containing the crystalline form and the solvent or solvents including, but not limited to, a suspension, solution, slurry, and emulsion.

The process can further include washing the obtained solid form.

Drying can be performed under ambient or reduced pressure. For example, drying can be performed under reduced pressure or under atmospheric pressure at a temperature of at about 40° C. to 60° C., or 70° C. to 80° C., or higher, depending on the volatility of the solvent that is being removed. The atmosphere for drying can be air or a partially or completely inert atmosphere, such as by using nitrogen.

Certain aspects and embodiments of the invention are further illustrated by the following examples, which are not intended to limit the scope of the claimed invention.

### EXAMPLE 1

Preparation of Crystalline Form II of Zolmitriptan

2 g of zolmitriptan was dissolved in 200 ml of dichloromethane at about 25° C. the solution was passed through a celite bed and the bed was washed with 10 ml of dichloromethane. The filtrate was distilled completely under reduced pressure at about 35° C. The obtained solid was dried at about 40° C. under reduced pressure to afford 1.6 g of the title compound.

### EXAMPLE 2

Preparation of Crystalline Form III of Zolmitriptan

10 g of zolmitriptan was suspended in 200 ml of toluene and heated to about 65° C. 35 ml of methanol was added slowly with stirring at the same temperature to form a clear solution and stirred for 20 minutes. The resultant solution was then cooled to about 25° C. and stirred for about 40 minutes. The separated solid was filtered and washed with 25 ml of toluene. The solid was dried at 45 to 50° C. under reduced pressure to yield 5.6 g of the title compound.

### EXAMPLE 3

Preparation of Solid Amorphous Zolmitriptan

3 g of zolmitriptan was dissolved in 170 ml of dichloromethane at about 39° C. and the solution was filtered through a filter paper followed by filtration through cloth. The filtrate was cooled to about 25 to 35° C. and maintained for 1 to 2 hours. The separated solid was filtered. The solid was dried under reduced pressure at 3540° C. to afford 1.1 g of the amorphous form of zolmitriptan.

1. Crystalline Form II of zolmitriptan.

2. The crystalline form of claim 1, having an X-ray diffraction pattern substantially in accordance with FIG. 1.

3. The crystalline form of claim 1, having an X-ray powder diffraction pattern comprising peaks at about 8.3, 11.8, 14.0, 17.3, 19.9, 22.1, 22.8, 23.7, and 25.3, ±0.2 degrees 2θ.

4. The crystalline form of claim 3, wherein the pattern further comprises peaks at about 27.0, 27.7, 29.5, and 36.2, ±0.2 degrees 2θ.

5. The crystalline form of claim 1, being prepared by a method comprising evaporation of solvent from a solution of zolmitriptan in a solvent comprising a halogenated hydrocarbon.

6. Crystalline Form III of zolmitriptan.

7. The crystalline form of claim 6, having an X-ray diffraction pattern substantially in accordance with FIG. 2.

8. The crystalline form of claim 6, having an X-ray powder diffraction pattern comprising peaks at about 8.3, 11.1, 14.8, 15.3, 17.5, 18.0, 18.4, 19.6, 23.7, and 25.5, ±0.2 degrees 2θ.

9. The crystalline form of claim 8, wherein the pattern further comprises peaks at about 13.2, 13.6, 23.3, 25.8, and 27.4, ±0.2 degrees 2θ.

10. The crystalline form of claim 6, being prepared by a method comprising suspending zolmitriptan in an aromatic hydrocarbon, adding a lower alcohol to form a solution, and crystallizing Form III of zolmitriptan.

11. Solid amorphous zolmitriptan.

12. The solid amorphous zolmitriptan of claim 11, having an X-ray powder diffraction pattern substantially in accordance with FIG. 3.

13. The solid amorphous zolmitriptan of claim 11, being prepared by a method comprising cooling a solution of zolmitriptan to a temperature where precipitation of amorphous zolmitriptan occurs.

14. The solid amorphous zolmitriptan of claim 11, being prepared by a method comprising cooling a solution of zolmitriptan in a halogenated hydrocarbon solvent to a temperature where precipitation of amorphous zolmitriptan occurs.
